(19) United States
(12) Patent Application Publication
(10) Pub. No.: US 2026/0224860 A1
(43) Pub. Date: Aug. 6, 2026

(54) GUIDE WIRE INSERTION ASSISTING TOOL

(71) Applicant: TERUMO KABUSHIKI KAISHA, Tokyo (JP)

(72) Inventor: Shinichi MIZUNO, Nakakoma-gun (JP)

(73) Assignee: TERUMO KABUSHIKI KAISHA, Tokyo (JP)

(21) Appl. No.: 19/550,728

(22) Filed: Feb. 26, 2026

Related U.S. Application Data
(63) Continuation of application No. PCT/JP2024/027405, filed on Jul. 31, 2024.

(30) Foreign Application Priority Data
Sep. 1, 2023 (JP) 2023-142134

(51) Int. Cl. A61M 25/09; A61M 25/00; A61M 39/06

(52) U.S. Cl. CPC A61M 25/09041; A61M 25/0097; A61M 39/06; A61M 2039/0626; A61M 2039/0633; A61M 2209/04

(57) ABSTRACT

In a guide wire insertion assisting tool, in a state in which a hub fitting portion of a joint member is fitted to a proximal portion of a catheter hub of a catheter assembly, a guide member is inserted into a lumen of the joint member, and the guide member and a guide fitting portion are fitted to each other, the guide member opens a valve body of the catheter assembly with relative displacement in a distal direction with respect to the joint member without displacing an opening member to the position at which the valve body is opened by the opening member.

### CROSS-REFERENCE TO RELATED APPLICATIONS

This is a bypass continuation of PCT Application No. PCT/JP2024/027405, filed on Jul. 31, 2024, which claims priority to Japanese Patent Application No. 2023-142134, filed on Sep. 1, 2023. The entire contents of these applications are hereby incorporated by reference herein.

### BACKGROUND

The present disclosure relates to a guide wire insertion assisting tool for inserting a guide wire into a catheter assembly.

A catheter assembly is used as an indwelling needle for administering an infusion solution (medicinal solution) into a blood vessel of a patient (a living body). A catheter assembly includes a catheter shaft and a catheter hub. The catheter hub is disposed at the proximal portion of the catheter shaft.

Meanwhile, in medical practice, there is a need to use a catheter assembly as an introduction needle for a guide wire. In this case, the guide wire is inserted into the lumen of the catheter hub from the proximal opening of the catheter hub. The inserted guide wire is inserted into a blood vessel through the catheter shaft.

Japanese Patent 4409329 B2 discloses a guide wire insertion assisting tool for inserting a guide wire into a catheter shaft from the outside through a catheter hub.

### SUMMARY

In a case where a catheter assembly includes a valve body and an opening member, the valve body is disposed in the lumen of the catheter hub. In the lumen of the catheter hub, the opening member is disposed between the valve body and the proximal opening of the catheter hub. The opening member is displaced in the distal direction, to open the valve body. Also, as the valve body and the opening member are disposed in the lumen, the inner surface of the catheter hub is formed in an uneven shape. Because of this, when a guide wire is inserted into the lumen of the catheter hub, there is a possibility that the end of the guide wire will be caught by the opening member or the uneven inner surface of the catheter hub. Also, there is a possibility that the end of the guide wire will come into contact with the valve body and damage the valve body. Therefore, there is a demand for a catheter assembly that includes a valve body and an opening member, and can be used as an introduction needle for a guide wire.

Certain embodiments of the present disclosure aim to solve the above problems.

(1) An embodiment of the present invention is a guide wire insertion assisting tool for inserting a guide wire into a catheter assembly including a catheter shaft, a catheter hub that is hollow and is disposed at a proximal portion of the catheter shaft, a valve body disposed in a lumen of the catheter hub, and an opening member that is disposed between the valve body in the lumen of the catheter hub and a proximal opening of the catheter hub and is displaced in a distal direction to open the valve body, the guide wire insertion assisting tool including: a joint member that is hollow, is connected to a proximal portion of the catheter hub, and is inserted into the lumen of the catheter hub from the proximal opening of the catheter hub; and a guide member that is inserted into a lumen of the joint member from a proximal opening of the joint member, and has a guide hole for guiding the guide wire. In the guide wire insertion assisting tool, the joint member includes a hub fitting portion that is inserted into the lumen of the catheter hub from the proximal opening of the catheter hub and is fitted to the proximal portion of the catheter hub, and a guide fitting portion that is fitted to the guide member inserted into the lumen of the joint member. When the joint member is connected to the proximal portion of the catheter hub, the joint member is inserted into the lumen of the catheter hub, without coming into contact with the opening member. When the hub fitting portion is fitted to the proximal portion of the catheter hub and the guide member is fitted to the guide fitting portion, a distal portion of the guide member protrudes from a distal opening of the joint member in the distal direction and is inserted into the valve body. When the guide member is inserted into the lumen of the joint member and the guide member is fitted to the guide fitting portion while the hub fitting portion is fitted to the proximal portion of the catheter hub, the guide member opens the valve body with relative displacement in the distal direction with respect to the joint member, without displacing the opening member to a position at which the valve body is opened.

With this configuration, the joint member does not come into contact with the opening member when the joint member is connected to the proximal portion of the catheter hub. Also, the guide member inserted into the joint member opens the valve body, without displacing the opening member to the position where the valve body is opened. Further, the proximal portion of the catheter hub and the hub fitting portion are fitted to each other, and the guide member is fitted to the guide fitting portion, so that the distal portion of the guide member can be brought closer to the central axis of the catheter shaft.

Thus, when the guide wire is inserted into the guide member from outside, the guide wire can be guided into the lumen of the catheter shaft, without the distal end of the guide wire being caught by the opening member and the uneven inner surface of the catheter hub. Furthermore, the distal end of the guide wire does not come into contact with the valve body. In other words, the guide wire can be inserted directly into the lumen of the catheter shaft, without damaging the valve body.

According this embodiment, the catheter assembly including the valve body and the opening member can be used as an introduction needle for the guide wire. Also, when the guide member is inserted into the joint member, the opening member does not advance in the distal direction and does not open the valve body. Thus, it is possible to prevent blood from leaking out of the catheter hub.

(2) In the guide wire insertion assisting tool according to (1), the guide wire may be inserted into the guide hole from a hollow guide wire supply tool through a proximal opening of the guide member, and the guide member may include an inner fitting portion that is disposed at a proximal portion of the guide member and is fitted to an inner surface of the guide wire supply tool.

With this configuration, the inner surface of the guide wire supply tool and the inner fitting portion of the guide member are fitted to each other, so that the guide wire can be effectively inserted into the guide hole.

(3) In the guide wire insertion assisting tool according to (2), the joint member may include an outer fitting portion that is disposed at a proximal portion of the joint member and is fitted to an outer surface of the guide wire supply tool.

With this configuration, the outer surface of the guide wire supply tool and the outer fitting portion of the joint member are fitted to each other, so that the guide wire supply tool can be effectively connected to the guide wire insertion assisting tool.

(4) In the guide wire insertion assisting tool according to (3), the inner fitting portion may be disposed on an outer surface of the proximal portion of the guide member, the outer fitting portion may be disposed on an inner surface of the proximal portion of the joint member, and the inner fitting portion may be fitted to an inner surface of a distal portion of the guide wire supply tool and the outer fitting portion may be fitted to an outer surface of the distal portion of the guide wire supply tool, so that the inner fitting portion and the outer fitting portion nip the distal portion of the guide wire supply tool.

With this configuration, the inner fitting portion and the inner surface of the distal portion of the guide wire supply tool are fitted in surface contact with each other, and the outer fitting portion and the outer surface of the distal portion of the guide wire supply tool are fitted in surface contact with each other. Thus, the guide wire supply tool can be stably connected to the guide wire insertion assisting tool. Also, as the inner fitting portion and the outer fitting portion nip the distal portion of the guide wire supply tool, it is possible to prevent the occurrence of axial displacement of the guide member with respect to the joint member.

(5) In the guide wire insertion assisting tool according to any one of (1) to (4), the hub fitting portion may be a Luer taper that is formed on an outer surface of the joint member and is fitted to an inner surface of the catheter hub, the guide member may have a tapered shape that is tapered in the distal direction, and the guide fitting portion may be a taper that is formed on an inner surface of the joint member and is fitted to an outer surface of the guide member.

With this configuration, the Luer taper, which is the hub fitting portion of the joint member, is fitted to the inner surface of the catheter hub, so that the joint member can be coaxially connected to the catheter hub. Also, the taper, which is the guide fitting portion, and the outer surface of the guide member are fitted to each other, so that the guide member can be coaxially inserted into the joint member, and the insertion length of the guide member in the distal direction with respect to the catheter hub can be limited. Thus, the guide wire can be more effectively guided into the lumen of the catheter shaft.

According to an embodiment, the joint member does not come into contact with the opening member when the joint member is connected to the proximal portion of the catheter hub. Also, the guide member inserted into the joint member opens the valve body, without displacing the opening member to the position where the valve body is opened. Further, the proximal portion of the catheter hub and the hub fitting portion are fitted to each other, and the guide member is fitted to the guide fitting portion, so that the distal portion of the guide member can be brought closer to the central axis of the catheter shaft.

Thus, when the guide wire is inserted into the guide member from outside, the guide wire can be guided into the lumen of the catheter shaft, without the distal end of the guide wire being caught by the opening member and the uneven inner surface of the catheter hub. Furthermore, the distal end of the guide wire does not come into contact with the valve body. In other words, the guide wire can be inserted directly into the lumen of the catheter shaft, without damaging the valve body.

Accordingly, in this embodiment, the catheter assembly including the valve body and the opening member can be used as an introduction needle for the guide wire. Also, when the guide member is inserted into the joint member, the opening member does not advance in the distal direction and does not open the valve body. Thus, it is possible to prevent blood from leaking out of the catheter hub.

### BRIEF DESCRIPTION OF THE DRAWINGS

FIG. 1 is a cross-sectional view of a catheter assembly.

FIG. 2 is a cross-sectional view illustrating a state in which an insertion assisting tool according to the present embodiment is connected to the catheter assembly.

FIG. 3 is a cross-sectional view illustrating a state in which a needle member is removed from the catheter assembly.

FIG. 4 is a cross-sectional view illustrating a state in which a joint member is connected to the catheter assembly shown in FIG. 3.

FIG. 5 is a cross-sectional view illustrating a state in which a guide member is inserted into a lumen of the joint member shown in FIG. 4.

FIG. 6 is a cross-sectional view illustrating a state in which the distal portion of a supply tool is nipped between the proximal portion of the joint member and the proximal portion of the guide member shown in FIG. 5.

FIG. 7 is a cross-sectional view illustrating a state in which the proximal portion of the guide member and the distal portion of the supply tool are fitted to each other.

### DETAILED DESCRIPTION

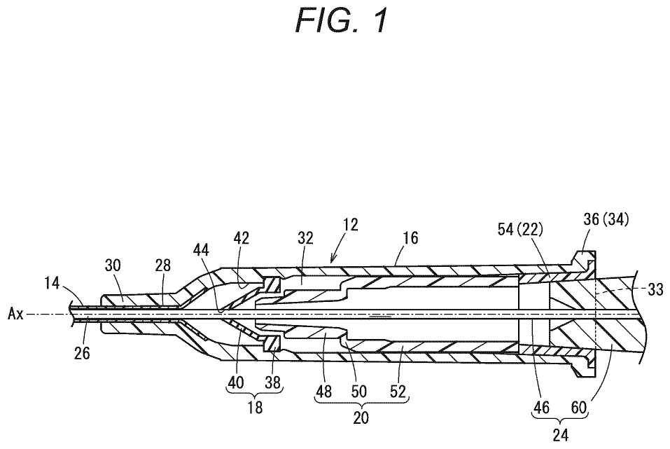

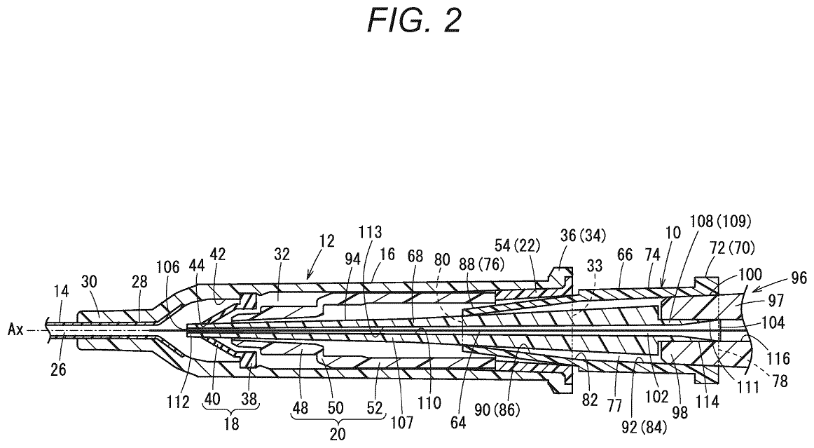

FIG. 1 is a cross-sectional view of a catheter assembly 12 in which a guide wire insertion assisting tool 10 according to the present embodiment (an insertion assisting tool 10 according to the present embodiment) (see FIG. 2) is adopted. The insertion assisting tool 10 according to the present embodiment is connected to the catheter assembly 12.

The catheter assembly 12 is configured as an indwelling needle for administering an infusion solution (medicinal solution) into a blood vessel of a patient (a living body) (not shown). The catheter assembly 12 includes a catheter shaft 14, a catheter hub 16, a valve body 18, an opening member 20, a restricting member 22, and a needle member 24.

The catheter shaft 14 is a tubular member having flexibility. The catheter shaft 14 is continuously insertable into a patient's blood vessel. The catheter shaft 14 has a lumen 26. The lumen 26 extends on the central axis of the catheter shaft 14. Note that the central axis of the catheter shaft 14 is coaxial with the central axis (axis line Ax) of the catheter assembly 12. The material constituting the catheter shaft 14 is a resin material, for example.

In the description below, in the catheter assembly 12, the direction along the axis line Ax toward the catheter shaft 14 (the leftward direction in FIG. 1) will be referred to as a distal direction. Also, a direction away from the catheter shaft 14 along the axis line Ax (the rightward direction in FIG. 1) will be referred to as a proximal direction.

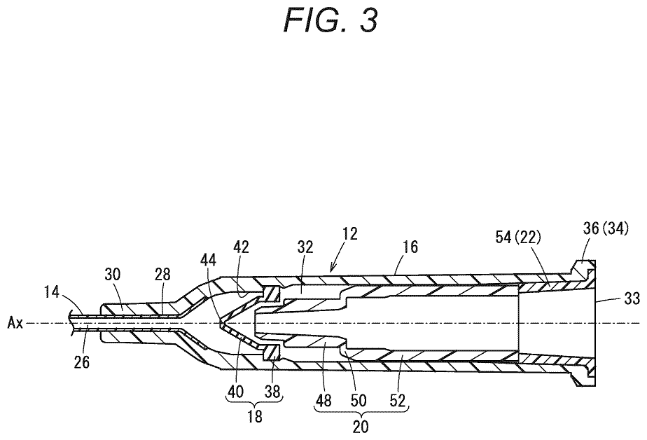

The catheter hub 16 is disposed at a proximal portion 28 of the catheter shaft 14. The proximal portion 28 of the catheter shaft 14 is connected to a distal portion 30 of the catheter hub 16. The catheter hub 16 is a tubular member. The catheter hub 16 has a lumen 32. The lumen 32 of the catheter hub 16 extends along the axis line Ax. The lumen 32 of the catheter hub 16 communicates with the lumen 26 of the catheter shaft 14. The lumen 32 of the catheter hub 16 communicates with the outside through a proximal opening 33 (see FIG. 3) formed on the proximal side. The central axis of the catheter hub 16 is coaxial with the axis line Ax.

The distal portion 30 of the catheter hub 16 is coaxially joined to the proximal portion 28 of the catheter shaft 14. A flange 36 is disposed at a proximal portion 34 of the catheter hub 16. The flange 36 protrudes radially outward from the catheter hub 16. The catheter hub 16 is preferably formed with a harder material than that of the catheter shaft 14. The material constituting the catheter hub 16 is a resin material, for example.

The valve body 18 is disposed in the lumen 32 of the catheter hub 16. The valve body 18 is a hemostasis valve for preventing blood from leaking into a portion of the lumen 32 of the catheter hub 16 on the proximal side of the valve body 18. That is, the valve body 18 prevents blood from leaking from the lumen 32 of the catheter hub 16. The material constituting the valve body 18 is an elastic material such as rubber, for example. The valve body 18 includes an annular portion 38 and a valve main body 40. The annular portion 38 is fixed to an inner surface 42 forming the lumen 32 of the catheter hub 16. The valve main body 40 protrudes in the distal direction from the annular portion 38. A slit 44 is formed at the distal portion of the valve main body 40. An inner needle 46 constituting the needle member 24 is inserted through the slit 44.

The opening member 20 is disposed in the lumen 32 of the catheter hub 16 on the proximal side of the valve body 18. The opening member 20 is a hollow plug. The opening member 20 is displaced in the distal direction with respect to the catheter hub 16, to open the valve body 18.

The opening member 20 includes a cylindrical pressing portion 48, an annular wall portion 50, and a cylindrical sidewall portion 52. The pressing portion 48 is the distal portion of the opening member 20. The pressing portion 48 can be inserted into the lumen of the annular portion 38 of the valve body 18. The pressing portion 48 opens the valve body 18 by pushing and expanding the slit 44 of the valve body 18 when the opening member 20 is displaced in the distal direction with respect to the catheter hub 16. The wall portion 50 protrudes radially outward from the proximal portion of the pressing portion 48. The sidewall portion 52 extends in the proximal direction from the outer edge of the wall portion 50. The outer diameter of the sidewall portion 52 is slightly smaller than the inner diameter of the catheter hub 16.

Note that, in a case where the catheter assembly 12 is used as an indwelling needle, a connector provided at the distal portion of an infusion line (not shown) is connected to the proximal portion 34 of the catheter hub 16. When the connector is pushed in the distal direction and comes into contact with the opening member 20, the opening member 20 moves in the distal direction.

The restricting member 22 is disposed in the lumen 32 of the catheter hub 16 on the proximal side of the opening member 20. The restricting member 22 is fitted to the inner surface 42 and the proximal portion 34 of the catheter hub 16 while being located on the proximal side of the opening member 20, and thus, is fixed to the catheter hub 16. The restricting member 22 restricts displacement of the opening member 20 in the proximal direction. Note that, in the catheter assembly 12, the restricting member 22 is not an essential component.

The restricting member 22 has a cylindrical portion 54. The cylindrical portion 54 is disposed along the axis line Ax in the lumen 32 of the catheter hub 16. The cylindrical portion 54 is in contact with the sidewall portion 52 of the opening member 20. For example, an extension portion (not shown) extending from the sidewall portion 52 in the proximal direction is inserted into an insertion hole (not shown) formed in the axial direction in the cylindrical portion 54, so that the cylindrical portion 54 and the sidewall portion 52 come into contact with each other.

The needle member 24 includes the inner needle 46 and a needle hub 60. The inner needle 46 is a tubular member that has such rigidity that it can puncture the skin of the patient. The inner needle 46 has a lumen (not shown). The inner needle 46 is inserted into the lumen 26 of the catheter shaft 14 and the lumen 32 of the catheter hub 16. The distal portion of the catheter shaft 14 is tapered. When the inner needle 46 is inserted through the lumen 26 of the catheter shaft 14, the outer surface of the inner needle 46 comes into contact with the inner surface of the distal portion of the catheter shaft 14. The material constituting the inner needle 46 is a metal material such as stainless steel, for example. The entire length of the inner needle 46 is sufficiently longer than that of the catheter shaft 14. A needle tip (not shown) that is the end of the inner needle 46 protrudes from a distal opening (not shown) of the catheter shaft 14.

The needle hub 60 is fixed to the proximal portion of the inner needle 46. The needle hub 60 has such a size that a user can grip it. The user operates the double tube of the catheter shaft 14 and the inner needle 46 while gripping the needle hub 60. The needle hub 60 is desirably formed with a harder material than the inner needle 46.

The needle member 24 is inserted into the lumen 32 of the catheter hub 16 through the proximal opening 33 of the catheter hub 16. The needle member 24 is detachable in the proximal direction with respect to the catheter hub 16.

As illustrated in FIG. 2, the insertion assisting tool 10 according to the present embodiment is connected to the catheter assembly 12 in a case where the catheter assembly 12 is used as an introduction needle for a guide wire 64. The insertion assisting tool 10 can be connected to the catheter hub 16, instead of the needle member 24, in a state where the needle member 24 has been removed from the catheter assembly 12 (see FIG. 3). The insertion assisting tool 10 can insert the guide wire 64 into the catheter assembly 12, while being connected to the catheter hub 16.

Specifically, the insertion assisting tool 10 includes a joint member 66 and a guide member 68.

The joint member 66 is a tubular member. The joint member 66 is connected to the proximal portion 34 of the catheter hub 16. Specifically, the joint member 66 is fitted to the inner surface of the restricting member 22, and thus, is connected to the proximal portion 34 of the catheter hub 16. Note that, in a case where the catheter assembly 12 does not include the restricting member 22, the joint member 66 is directly fitted (locked) to the inner surface of the proximal portion 34 of the catheter hub 16. The joint member 66 is inserted into the lumen 32 of the catheter hub 16 from the proximal opening 33 of the catheter hub 16, without coming into contact with the opening member 20. Therefore, the distal portion of the joint member 66 has such an outer diameter that it can be inserted into the opening member 20. Also, the joint member 66 is connected to the proximal portion 34 of the catheter hub 16, without coming into contact with the opening member 20. The material constituting the joint member 66 is a resin material, for example.

A flange 72 protruding radially outward is formed at a proximal portion 70 of the joint member 66.

Of an outer surface 74 of the joint member 66, the portion between the intermediate portion in the axial direction of the joint member 66 and the flange 72 is formed in a straight shape. A Luer taper 76 tapered in the distal direction is formed on the outer surface 74 of the joint member 66 at the portion between the intermediate portion and the distal end of the joint member 66 in the axial direction.

The joint member 66 has a lumen 77. The lumen 77 is formed in the axial direction of the joint member 66. When the joint member 66 is connected to the proximal portion 34 of the catheter hub 16, the lumen 77 is coaxial with the axis line Ax. A proximal opening 78 is formed at the proximal end of the joint member 66. A distal opening 80 is formed at the distal end of the joint member 66. The lumen 77 of the joint member 66 communicates with the outside through the proximal opening 78 and the distal opening 80.

The inner surface 82 of the joint member 66 forms the lumen 77. A Luer taper 84 is formed on an inner surface 82 of the joint member 66 at the portion between the intermediate portion of the joint member 66 in the axial direction and the proximal opening 78. A taper 86 tapered in the distal direction is formed on the inner surface 82 of the joint member 66 at the portion between the intermediate portion of the joint member 66 in the axial direction and the distal opening 80.

The joint member 66 includes a hub fitting portion 88, a guide fitting portion 90, and an outer fitting portion 92.

The hub fitting portion 88 is a portion on the distal side of the outer surface 74 of the joint member 66. That is, the hub fitting portion 88 is a portion of the Luer taper 76 formed on the outer surface 74 of the joint member 66. When the joint member 66 is inserted into the lumen 32 of the catheter hub 16 from the proximal opening 33 of the catheter hub 16, the hub fitting portion 88 is fitted to the inner surface of the proximal portion 34 of the catheter hub 16 via the restricting member 22.

The guide fitting portion 90 is a portion of the inner surface 82 of the joint member 66 on the distal side. That is, the guide fitting portion 90 is a portion of the taper 86 formed on the inner surface 82 of the joint member 66. The guide fitting portion 90 is fitted to an outer surface 94 of the guide member 68 when the guide member 68 is inserted into the lumen 32 of the joint member 66.

The outer fitting portion 92 is a portion of the inner surface 82 of the joint member 66 on the proximal side. That is, the outer fitting portion 92 is a portion of the Luer taper 84 formed on the inner surface 82 of the joint member 66. The outer fitting portion 92 is fitted in surface contact with an outer surface 100 of a distal portion 98 of a connector 97 constituting part of a supply tool 96 (a guide wire supply tool) that will be described later.

The guide member 68 is a tubular member that is longer than the joint member 66. The guide member 68 is inserted into the lumen 77 of the joint member 66 from the proximal opening 78 of the joint member 66. The guide member 68 has such an outer diameter that it can be inserted into the opening member 20. The guide member 68 has a guide hole 102 for guiding the guide wire 64. The guide hole 102 has an inner diameter that allows the guide wire 64 to pass through the hole. The guide hole 102 is formed on the central axis of the guide member 68. A proximal opening 104 is formed at the proximal end of the guide member 68. A distal opening 106 is formed at the distal end of the guide member 68. The guide hole 102 of the guide member 68 communicates with the outside through the proximal opening 104 and the distal opening 106. The material constituting the guide member 68 is a resin material, for example.

Specifically, the guide member 68 includes a guide main body 107 and a protruding portion 109. The guide main body 107 is a tubular portion that is tapered in the distal direction. The outer surface 94 of the guide main body 107 has a tapered shape that is tapered in the distal direction. Accordingly, the outer diameter of the guide main body 107 is greater at a portion closer to the proximal end. The protruding portion 109 protrudes in the proximal direction from the proximal end of the guide main body 107. The protruding portion 109 is a tubular portion protruding straight in the proximal direction from the proximal end of the guide main body 107. The outer diameter of the protruding portion 109 is smaller than the outer diameter of the proximal end of the guide main body 107. The protruding portion 109 is a proximal portion 108 of the guide member 68. The guide hole 102 penetrates the guide main body 107 and the protruding portion 109. The proximal opening 104 of the guide hole 102 is formed at the proximal end of the protruding portion 109. The distal opening 106 of the guide hole 102 is formed at the distal end of the guide main body 107.

An inner surface 110 of the guide member 68 forms the guide hole 102. Of the inner surface 110 of the guide member 68, the inner surface of the proximal portion 108 has a tapered portion 111 that is tapered in the distal direction from the proximal opening 104. Of the inner surface 110 of the guide member 68, the portion between a tapered portion 111 and the distal opening 106 has a straight portion 113 that extends straight.

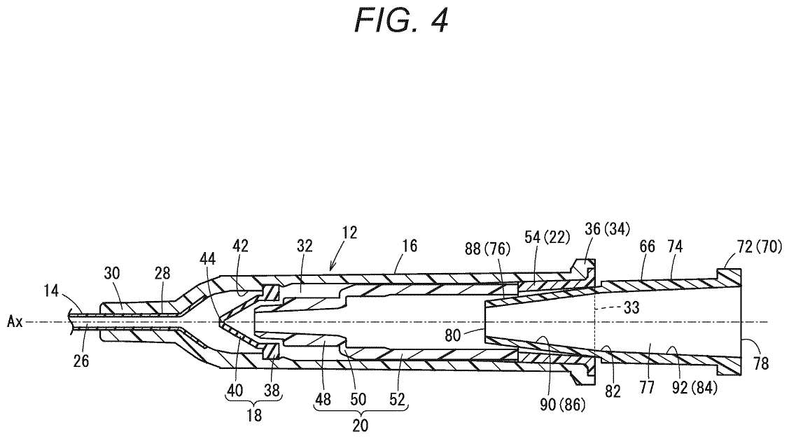

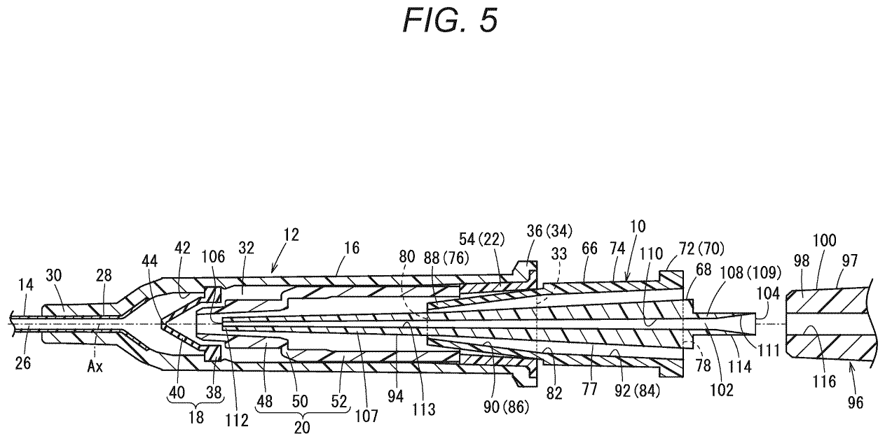

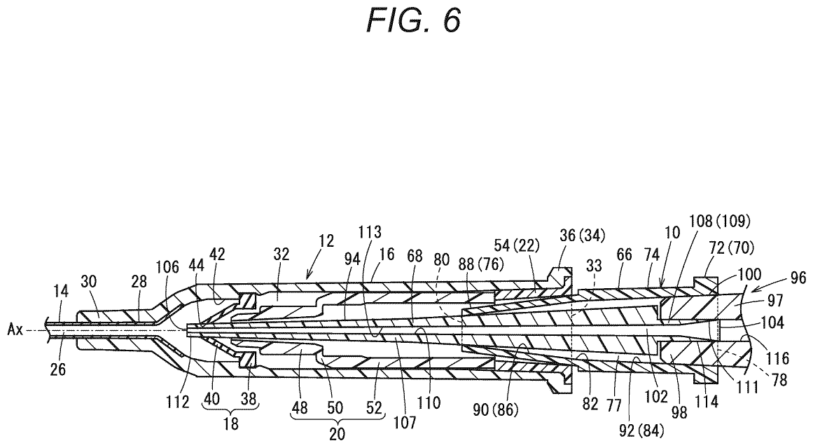

When the guide member 68 is inserted into the lumen 77 of the joint member 66 from the proximal opening 78 of the joint member 66 (see FIG. 5) while the hub fitting portion 88 is fitted to the proximal portion 34 of the catheter hub 16 (see FIG. 4), the guide member 68 is relatively displaced in the distal direction with respect to the joint member 66. When the guide member 68 is displaced in the distal direction, a distal portion 112 of the guide member 68 protrudes in the distal direction from the distal opening 80 of the joint member 66. When the guide member 68 is further displaced in the distal direction, the outer surface 94 of the guide main body 107 and the guide fitting portion 90 are fitted to each other (see FIG. 6). In this case, the distal portion 112 of the guide member 68 is inserted into the valve body 18, without displacing the opening member 20 to the position where the valve body 18 is opened. As a result, the distal portion 112 of the guide member 68 is inserted through the slit 44, to open the valve body 18.

The guide member 68 includes an inner fitting portion 114. The inner fitting portion 114 is the outer surface of the protruding portion 109. The supply tool 96 includes the connector 97. The inner fitting portion 114 is fitted in surface contact with an inner surface 116 of the distal portion 98 of the connector 97.

The guide wire 64 is inserted into the guide hole 102 from the connector 97 through the proximal opening 104 of the guide member 68. The connector 97 is a hollow member. The distal portion 98 of the connector 97 can be inserted between the outer fitting portion 92 and the inner fitting portion 114. When the distal portion 98 of the connector 97 is inserted between the outer fitting portion 92 and the inner fitting portion 114, the inner fitting portion 114 is fitted to the inner surface 116 of the distal portion 98 of the connector 97, and the outer fitting portion 92 is fitted to the outer surface 100 of the distal portion 98 of the connector 97. As a result, the inner fitting portion 114 and the outer fitting portion 92 nip the distal portion 98 of the connector 97.

Next, insertion of the guide wire 64 into the catheter assembly 12 by the insertion assisting tool 10 is described. Here, a first method of use and a second method of use are described.

The first method of use is now described. By the first method of use, first, while the catheter shaft 14 of the catheter assembly 12 (see FIG. 1), which is an indwelling needle, is inserted into a blood vessel of a patient, the needle member 24 is pulled out in the proximal direction. At this point of time, when the tip of the inner needle 46 moves in the proximal direction with respect to the valve body 18, the slit 44 is closed, and the valve body 18 becomes blocked. As a result, the valve body 18 can prevent blood from leaking from the lumen 32 of the catheter hub 16 to the outside. When the needle member 24 is further pulled out in the proximal direction, the needle member 24 is detached from the catheter assembly 12 (see FIG. 3).

Next, as illustrated in FIG. 4, the joint member 66 is inserted into the lumen 32 through the proximal opening 33 (see FIG. 3) of the catheter hub 16. At this point of time, the joint member 66 is inserted into the lumen 32 of the catheter hub 16, without coming into contact with the opening member 20. When the joint member 66 is further displaced in the distal direction, the hub fitting portion 88 is fitted to the inner surface of the proximal portion 34 of the catheter hub 16 via the restricting member 22.

Next, as illustrated in FIG. 5, the guide member 68 is inserted into the lumen 77 through the proximal opening 78 of the joint member 66. The guide member 68 is displaced in the distal direction through the lumen 77 of the joint member 66. When the guide member 68 is further displaced in the distal direction, the distal portion 112 of the guide member 68 protrudes in the distal direction from the distal opening 80 of the joint member 66.

When the guide member 68 is further displaced in the distal direction, the distal portion 112 of the guide member 68 is inserted into the lumen of the valve body 18, without coming into contact with the opening member 20. When the guide member 68 is further displaced in the distal direction, the distal portion 112 of the guide member 68 is inserted through the slit 44, to open the valve body 18, as illustrated in FIG. 6.

Also, the outer surface 94 of the guide main body 107 is fitted to the guide fitting portion 90. As the guide fitting portion 90 is fitted to the outer surface 94 of the guide main body 107, movement of the guide member 68 in the distal direction is restricted, and the distal portion 112 of the guide member 68 is moved toward the axis line Ax. As a result, the distal opening 106 of the guide member 68 and the lumen 26 of the catheter shaft 14 face each other in the axial direction.

Next, the distal portion 98 of the connector 97 of the supply tool 96 is inserted between the outer fitting portion 92 and the inner fitting portion 114. As a result, the outer fitting portion 92 and the inner fitting portion 114 nip the distal portion 98 of the connector 97 in a radial direction.

Next, as illustrated in FIG. 2, the guide wire 64 is inserted into the guide hole 102 from the supply tool 96 through the proximal opening 104 of the guide member 68. As described above, the tapered portion 111 and the straight portion 113 are formed on the inner surface 110 of the guide member 68. That is, the inner surface 110 of the guide member 68 has no unevenness. Also, the distal opening 106 of the guide member 68 and the lumen 26 of the catheter shaft 14 face each other in the axial direction. Accordingly, the guide wire 64 inserted into the guide hole 102 is inserted into the lumen 26 of the catheter shaft 14 through the distal opening 106 of the guide member 68, without the distal end of the guide wire 64 being caught anywhere. Thus, the guide wire 64 is inserted into the blood vessel of the patient via the catheter shaft 14.

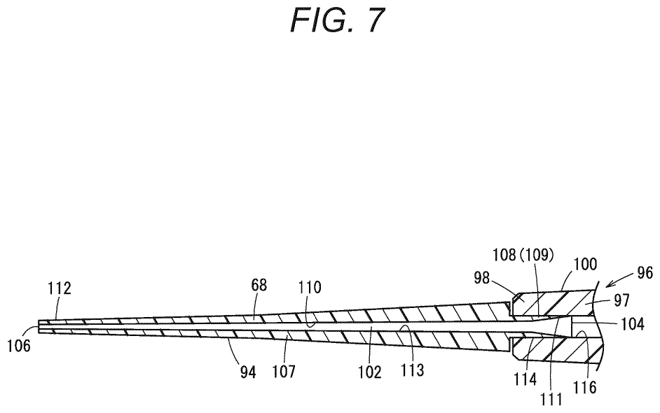

The second method of use is now described. As illustrated in FIG. 4, the second method of use is the same as the first method of use in the steps up to the step of connecting the proximal portion 34 of the catheter hub 16 and the joint member 66. By the second method of use, the inner fitting portion 114 of the guide member 68 and the inner surface 116 of the distal portion 98 of the connector 97 are fitted to each other in advance, as illustrated in FIG. 7. Alternatively, after the step of connecting the proximal portion 34 of the catheter hub 16 and the joint member 66, the inner fitting portion 114 of the guide member 68 and the inner surface 116 of the distal portion 98 of the connector 97 may be fitted to each other. After that, the guide member 68 fitted to the connector 97 is inserted into the lumen 77 of the joint member 66, as illustrated in FIG. 6.

The present embodiment provides the following effects.

As illustrated in FIG. 4, when the joint member 66 is connected to the proximal portion 34 of the catheter hub 16, the joint member 66 does not come into contact with the opening member 20. Also, as illustrated in FIG. 6, the guide member 68 inserted into the joint member 66 opens the valve body 18, without displacing the opening member 20 to the position where the valve body 18 is opened. Further, the proximal portion 34 of the catheter hub 16 and the hub fitting portion 88 are fitted to each other, and the guide member 68 is fitted to the guide fitting portion 90, so that the distal portion 112 of the guide member 68 can be brought closer to the central axis (axis line Ax) of the catheter shaft 14.

Thus, as illustrated in FIG. 2, when the guide wire 64 is inserted into the guide member 68 from the connector 97 of the supply tool 96, the guide wire 64 can be guided into the lumen 26 of the catheter shaft 14, without the distal end of the guide wire 64 being caught by the opening member 20 and the inner surface 42 of the catheter hub 16. Furthermore, the distal end of the guide wire 64 does not come into contact with the valve body 18. In other words, the guide wire 64 can be inserted directly into the lumen 26 of the catheter shaft 14, without damaging the valve body 18.

Accordingly, in the present embodiment, the catheter assembly 12 including the valve body 18 and the opening member 20 can be used as an introduction needle for the guide wire 64. Also, when the guide member 68 is inserted into the joint member 66, the opening member 20 does not advance in the distal direction and does not greatly open the valve body 18. Thus, it is possible to prevent blood from leaking out of the catheter hub 16 at once.

As illustrated in FIG. 6, the inner surface 116 of the connector 97 of the supply tool 96 and the inner fitting portion 114 of the guide member 68 are fitted to each other, so that the guide wire 64 (see FIG. 2) can be effectively inserted into the guide hole 102.

Also, the outer surface 100 of the connector 97 of the supply tool 96 and the outer fitting portion 92 of the joint member 66 are fitted to each other, so that the connector 97 of the supply tool 96 can be effectively connected to the insertion assisting tool 10.

The inner fitting portion 114 and the inner surface 116 of the distal portion 98 of the connector 97 constituting the supply tool 96 are fitted in surface contact with each other, and the outer fitting portion 92 and the outer surface 100 of the distal portion 98 of the connector 97 are fitted in surface contact with each other. Thus, the connector 97 of the supply tool 96 can be stably connected to the insertion assisting tool 10. Also, as the inner fitting portion 114 and the outer fitting portion 92 nip the distal portion 98 of the connector 97, it is possible to prevent the occurrence of axial displacement of the guide member 68 with respect to the joint member 66.

The Luer taper 76, which is the hub fitting portion 88 of the joint member 66, is fitted to the inner surface 42 of the catheter hub 16, so that the joint member 66 can be coaxially connected to the catheter hub 16. Also, the taper 86, which is the guide fitting portion 90, and the outer surface 94 of the guide member 68 are fitted to each other, so that the guide member 68 can be coaxially inserted into the joint member 66, and the insertion length of the guide member 68 in the distal direction with respect to the catheter hub 16 can be limited. Thus, the guide wire 64 can be more effectively guided into the lumen 26 of the catheter shaft 14.

Note that the present invention is not limited to the above disclosure and can take various configurations without departing from the gist of the present invention.

1. A guide wire insertion assisting tool for inserting a guide wire into a catheter assembly that comprises a catheter shaft, a catheter hub that is hollow and is disposed at a proximal portion of the catheter shaft, a valve body disposed in a lumen of the catheter hub, and an opening member that is disposed in the lumen of the catheter hub between the valve body and a proximal opening of the catheter hub and is displaceable in a distal direction to open the valve body, the guide wire insertion assisting tool comprising:
a joint member that is hollow, is connectable to a proximal portion of the catheter hub, and is insertable into the lumen of the catheter hub from the proximal opening of the catheter hub; and
a guide member that is insertable into a lumen of the joint member from a proximal opening of the joint member, and has a guide hole for guiding the guide wire, wherein:
the joint member comprises a hub fitting portion that is configured to be inserted into the lumen of the catheter hub from the proximal opening of the catheter hub and to be fitted to the proximal portion of the catheter hub, and a guide fitting portion that is configured to be fitted to the guide member inserted into the lumen of the joint member,
the joint member is configured such that, when the joint member is connectable to the proximal portion of the catheter hub, the joint member is inserted into the lumen of the catheter hub without coming into contact with the opening member,
the guide member is configured such that, when the hub fitting portion is fitted to the proximal portion of the catheter hub and the guide member is fitted to the guide fitting portion, a distal portion of the guide member protrudes from a distal opening of the joint member in the distal direction and is inserted into the valve body, and,
the guide member is configured such that, when the guide member is inserted into the lumen of the joint member and the guide member is fitted to the guide fitting portion while the hub fitting portion is fitted to the proximal portion of the catheter hub, the guide member opens the valve body with relative displacement in the distal direction with respect to the joint member, without displacing the opening member to a position at which the valve body is opened by the opening member.

2. The guide wire insertion assisting tool according to claim 1, wherein:
the guide member comprises an inner fitting portion that is disposed at a proximal portion of the guide member and is configured to be fitted to an inner surface of a guide wire supply tool that is configured to supply the guide wire into the guide hole.

3. The guide wire insertion assisting tool according to claim 2, wherein:
the joint member comprises an outer fitting portion that is disposed at a proximal portion of the joint member and is configured to be fitted to an outer surface of the guide wire supply tool.

4. The guide wire insertion assisting tool according to claim 3, wherein:
the inner fitting portion is disposed at an outer surface of the proximal portion of the guide member,
the outer fitting portion is disposed at an inner surface of the proximal portion of the joint member, and
the inner fitting portion is configured to be fitted to an inner surface of a distal portion of the guide wire supply tool, and the outer fitting portion is configured to be fitted to an outer surface of the distal portion of the guide wire supply tool, to cause the inner fitting portion and the outer fitting portion to nip the distal portion of the guide wire supply tool.

5. The guide wire insertion assisting tool according to claim 1, wherein:
the hub fitting portion is a Luer taper that is formed on an outer surface of the joint member and is configured to be fitted to an inner surface of the catheter hub,
the guide member has a tapered shape that is tapered in the distal direction, and
the guide fitting portion is a taper that is formed on an inner surface of the joint member and is configured to be fitted to an outer surface of the guide member.

6. The guide wire insertion assisting tool according to claim 2, wherein:
the hub fitting portion is a Luer taper that is formed on an outer surface of the joint member and is configured to be fitted to an inner surface of the catheter hub,
the guide member has a tapered shape that is tapered in the distal direction, and
the guide fitting portion is a taper that is formed on an inner surface of the joint member and is configured to be fitted to an outer surface of the guide member.

7. The guide wire insertion assisting tool according to claim 3, wherein:
the hub fitting portion is a Luer taper that is formed on an outer surface of the joint member and is configured to be fitted to an inner surface of the catheter hub,
the guide member has a tapered shape that is tapered in the distal direction, and
the guide fitting portion is a taper that is formed on an inner surface of the joint member and is configured to be fitted to an outer surface of the guide member.

8. The guide wire insertion assisting tool according to claim 4, wherein:
the hub fitting portion is a Luer taper that is formed on an outer surface of the joint member and is configured to be fitted to an inner surface of the catheter hub,
the guide member has a tapered shape that is tapered in the distal direction, and
the guide fitting portion is a taper that is formed on an inner surface of the joint member and is configured to be fitted to an outer surface of the guide member.

9. A set comprising:
a catheter assembly that comprises:
a catheter shaft,
a catheter hub that is hollow and is disposed at a proximal portion of the catheter shaft,
a valve body disposed in a lumen of the catheter hub, and
an opening member that is disposed in the lumen of the catheter hub between the valve body and a proximal opening of the catheter hub and is displaceable in a distal direction to open the valve body; and
guide wire insertion assisting tool for inserting a guide wire into the catheter assembly, the guide wire insertion assisting tool comprising:
a joint member that is hollow, is connectable to a proximal portion of the catheter hub, and is insertable into the lumen of the catheter hub from the proximal opening of the catheter hub, and
a guide member that is insertable into a lumen of the joint member from a proximal opening of the joint member, and has a guide hole for guiding the guide wire, wherein:
the joint member comprises a hub fitting portion that is configured to be inserted into the lumen of the catheter hub from the proximal opening of the catheter hub and to be fitted to the proximal portion of the catheter hub, and a guide fitting portion that is configured to be fitted to the guide member inserted into the lumen of the joint member,
the joint member is configured such that, when the joint member is connected to the proximal portion of the catheter hub, the joint member is inserted into the lumen of the catheter hub without coming into contact with the opening member,
the guide member is configured such that, when the hub fitting portion is fitted to the proximal portion of the catheter hub and the guide member is fitted to the guide fitting portion, a distal portion of the guide member protrudes from a distal opening of the joint member in the distal direction and is inserted into the valve body, and,
the guide member is configured such that, when the guide member is inserted into the lumen of the joint member and the guide member is fitted to the guide fitting portion while the hub fitting portion is fitted to the proximal portion of the catheter hub, the guide member opens the valve body with relative displacement in the distal direction with respect to the joint member, without displacing the opening member to a position at which the valve body is opened by the opening member.

10. The set according to claim 1, further comprising:
a guide wire supply tool configured to supply the guide wire through a proximal opening of the guide member and into the guide hole, wherein:
the guide member comprises an inner fitting portion that is disposed at a proximal portion of the guide member and is configured to be fitted to an inner surface of the guide wire supply tool.

11. The set according to claim 10, wherein:
the joint member comprises an outer fitting portion that is disposed at a proximal portion of the joint member and is configured to be fitted to an outer surface of the guide wire supply tool.

12. The set according to claim 11, wherein:
the inner fitting portion is disposed at an outer surface of the proximal portion of the guide member,
the outer fitting portion is disposed at an inner surface of the proximal portion of the joint member, and
the inner fitting portion is configured to be fitted to an inner surface of a distal portion of the guide wire supply tool, and the outer fitting portion is configured to be fitted to an outer surface of the distal portion of the guide wire supply tool, to cause the inner fitting portion and the outer fitting portion to nip the distal portion of the guide wire supply tool.

13. The set according to claim 9, wherein:
the hub fitting portion is a Luer taper that is formed on an outer surface of the joint member and is configured to be fitted to an inner surface of the catheter hub,
the guide member has a tapered shape that is tapered in the distal direction, and
the guide fitting portion is a taper that is formed on an inner surface of the joint member and is configured to be fitted to an outer surface of the guide member.

14. The set according to claim 10, wherein:
the hub fitting portion is a Luer taper that is formed on an outer surface of the joint member and is configured to be fitted to an inner surface of the catheter hub,
the guide member has a tapered shape that is tapered in the distal direction, and
the guide fitting portion is a taper that is formed on an inner surface of the joint member and is configured to be fitted to an outer surface of the guide member.

15. The set according to claim 11, wherein:
the hub fitting portion is a Luer taper that is formed on an outer surface of the joint member and is configured to be fitted to an inner surface of the catheter hub,
the guide member has a tapered shape that is tapered in the distal direction, and
the guide fitting portion is a taper that is formed on an inner surface of the joint member and is configured to be fitted to an outer surface of the guide member.

16. The set according to claim 12, wherein:
the hub fitting portion is a Luer taper that is formed on an outer surface of the joint member and is configured to be fitted to an inner surface of the catheter hub,
the guide member has a tapered shape that is tapered in the distal direction, and
the guide fitting portion is a taper that is formed on an inner surface of the joint member and is configured to be fitted to an outer surface of the guide member.

17. A method of inserting a guide wire into a catheter assembly using a guide wire insertion assisting tool, the method comprising:
Providing the catheter assembly, which comprises:
a catheter shaft,
a catheter hub that is hollow and is disposed at a proximal portion of the catheter shaft,
a valve body disposed in a lumen of the catheter hub, and
an opening member that is disposed in the lumen of the catheter hub between the valve body and a proximal opening of the catheter hub and is displaceable in a distal direction to open the valve body;
providing the guide wire insertion assisting tool, which comprises:
a joint member that is hollow and comprises a hub fitting portion and a guide fitting portion, and
a guide member that has a guide hole for guiding the guide wire, wherein:
inserting the hub fitting portion of the joint member into the lumen of the catheter hub from the proximal opening of the catheter hub and fitting the hub fitting portion of the joint member to a proximal portion of the catheter hub, such that the joint member does not contact the opening member;
inserting the guide member into a lumen of the joint member from a proximal opening of the joint member such that the guide fitting portion of the joint member is fitted to the guide member and such that a distal portion of the guide member protrudes from a distal opening of the joint member in the distal direction and is inserted into the valve body and the guide member opens the valve body with relative displacement in the distal direction with respect to the joint member without displacing the opening member to a position at which the valve body is opened by the opening member.
